(19) United States
(12) Patent Application Publication
(10) Pub. No.: US 2025/0110030 A1
(43) Pub. Date: Apr. 3, 2025

(54) Serviceable Dust Filter for Optical Based Gaseous Sensors

(71) Applicant: SeekOps Inc., Austin, TX (US)

(72) Inventors: Garrett Niall John, Buda, TX (US); John Thomas Dolecek, Austin, TX (US); Brendan James Smith, Lakeway, TX (US)

(21) Appl. No.: 18/899,876

(22) Filed: Sep. 27, 2024

Related U.S. Application Data
(60) Provisional application No. 63/541,744, filed on Sep. 29, 2023.

(51) Int. Cl. G01N 1/40; G01N 21/15

(52) U.S. Cl. CPC G01N 1/4077; G01N 21/15; G01N 2001/4088; G01N 2021/155; G01N 2021/157

(57) ABSTRACT

Systems, devices, and methods including an optical cell assembly, comprising: an optical core based trace gas sensor configured to measure trace gas concentrations; and an optical cell sub assembly including: a housing configured to house the optical core based trace gas sensor; and a field-replaceable filter media configured to be detachably attached to a portion of the housing and allow ambient trace gas to enter into the optical cell sub assembly.

### CROSS-REFERENCE TO RELATED APPLICATION

This application claims the priority benefit of U.S. Provisional Patent Application Ser. No. 63/541,744, filed Sep. 29, 2023, all of which are incorporated herein by reference in its entirety for all purposes.

### FIELD OF ENDEAVOR

Embodiments relate generally to gas measurement, and more particularly to filters for gas measurement instruments.

### BACKGROUND

Methane (CH4) is an odorless and colorless naturally occurring organic molecule, which is present in the atmosphere at average ambient levels of approximately 1.85 ppm as of 2018 and is projected to continually climb. Methane is a powerful greenhouse gas, a source of energy (i.e., methane is flammable), and an explosion hazard, and so detection of methane is of utility to scientists as well as engineers. While methane is found globally in the atmosphere, a significant amount is collected or “produced” through anthropogenic processes including exploration, extraction, and distribution of petroleum resources as a component in natural gas. Natural gas, an odorless and colorless gas, is a primary fuel used to produce electricity and heat. The main component of natural gas is typically methane, and the concentration of methane in a stream of natural gas can range from about 70% to 90%. The balance of the gas mixture in natural gas consists of longer chain hydrocarbons, including ethane, propane, and butane, typically found in diminishing mole fractions that depend on the geology of the earth from which the gas is extracted. Once extracted from the ground, natural gas is processed into a product that must comply with specifications for both transport, taxation, and end-use in burners; specification of processed ‘downstream’ natural gas product control for the composition of the gas, so as to protect transport lines from corrosion and ensure proper operation of burners and turbines. While extraction of natural gas is one of the main sources of methane in the atmosphere, major contributors of methane also include livestock farming (i.e., enteric fermentation) and solid waste and wastewater treatment (i.e., anaerobic digestion). Anaerobic digestion and enteric fermentation gas products consist primarily of methane and lack additional hydrocarbon species.

### SUMMARY

A system embodiment may include: an optical cell assembly comprising: an optical core based trace gas sensor configured to measure trace gas concentrations; and an optical cell sub assembly including: a housing configured to house the optical core based trace gas sensor; and a field-replaceable filter media configured to be detachably attached to a portion of the housing and allow ambient trace gas to enter into the optical cell sub assembly.

In another embodiment, the field-replaceable filter media may be a perforated outer sheath including a plurality of apertures.

In another embodiment, the perforated outer sheath may have a cylindrical shape with the plurality of apertures arranged along the circumference of the cylindrical shape.

In another embodiment, the system may further comprise one or more imbedded nozzles disposed on and through the surface of the optical cell sub assembly to allow for cleaning of the optics of the optical core based trace gas sensor within the optical cell sub assembly.

In another embodiment, 1, the system may further comprise: one or more extenders configured to connect between the optical cell assembly and one or more mounting attachments such that the optical cell assembly is located distal from the one or more mounting attachments; and the one or more mounting attachments configured to attach the system to an unmanned vehicle.

In another embodiment, the housing may include a plurality of apertures, and wherein the field-replaceable filter media is disposed on the plurality of apertures of the housing.

In another embodiment, the field-replaceable filter media may include a plurality of apertures, and wherein the plurality of apertures of the field-replaceable filter media may be respectively aligned with the plurality of apertures of housing.

In another embodiment, the housing may include an opening, and wherein the field-replaceable filter media may be inserted into the opening during use in the field.

In another embodiment, the field-replaceable filter media may include at least one of: a clip for securing the field-replaceable filter media to the opening and a protrusion for aligning the field-replaceable filter media with the opening.

In another embodiment, the field-replaceable filter media may include: a filter media and at least one of: a dust filter frame, a filter backing plate, and a dust filter gasket.

In another embodiment, the filter media may be made of the material having a plurality of pores.

In another embodiment, when the sizes of the pores are below a predetermined level, the system may further comprise an air movement system configured to pull ambient gas into the optical cell sub assembly and expel gas inside the optical cell sub assembly at a constant rate.

In another embodiment, the filter media may comprise at least one of: Nylon monofilament (NMO), polypropylene filter paper, a filter felt, a rayon fabric, a polyester fabric, a chemical resistant filter felt, a polyethersulfone (PES) membrane, and hydrophobic or oleophobic materials.

In another embodiment, the system may further comprise a pressure drop measurement module configured to measure a pressure drop across the field-replaceable filter media, and wherein the pressure drop measurement obtained from the pressure drop measurement module may be used to evaluate a buildup of particulates on the field-replaceable filter media.

In another embodiment, the system may further comprise a digital image processing module configured to perform a visual inspection of the field-replaceable filter media, and wherein the processed image obtained from the digital image processing module may be used to determine when the field-replaceable filter media needs to be replaced.

In another embodiment, the system may further comprise control electronics electronically connected to the optical core based trace gas sensor inside the optical cell sub assembly.

A method embodiment may include: applying a dust filter adhesive to a filter backing plate to form a bracket; attaching a filter media to the bracket to form a packing and filter media; attaching a dust filter frame to the packing and filter media to form a dust filter subassembly; and attaching a dust filter gasket to the dust filter subassembly to form a dust filter assembly.

In another embodiment, the method may further comprise: attaching the dust filter assembly into an opening of a housing of an optical core based trace gas sensor in a gas detection system during use in the field; and detaching the dust filter assembly from the housing to replace it with a new dust filter assembly during use in the field.

In another embodiment, the method may further comprise: evaluating a buildup of particulates on the dust filter assembly based on at least one of: a visual inspection of the dust filter assembly using a digital image processing module of the gas detection system; and a pressure drop measurement across the dust filter assembly using a pressure drop measurement module of the gas detection system.

Another system embodiment may include: an optical core based trace gas sensor configured to measure trace gas concentrations; and a housing having a cylindrical shape configured to house the optical core based trace gas sensor, wherein the housing includes a perforated outer sheath with a plurality of apertures arranged along at least a portion of circumference of the cylindrical shape, wherein the perforated outer sheath is configured to be detachably attached to a portion of the housing and allow ambient trace gas to enter into the optical core based trace gas sensor.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1 depicts an optical cell assembly of a gas detection system, according to one embodiment;

FIG. 2 depicts another optical cell assembly of a gas leak detection system, according to one embodiment;

FIG. 3A depicts a top view of a gas detection system, according to one embodiment;

FIG. 3B depicts a side view of the gas detection system of FIG. 3A, according to one embodiment;

FIG. 4A depicts a partial cut-away perspective view of an optical cell sub assembly having a field-replaceable filter media, according to one embodiment;

FIG. 4B depicts a partial cut-away perspective view of an optical cell sub assembly having a field-replaceable filter media, according to another embodiment;

FIGS. 5A-5B depict perspective views of a field-replaceable filter media for an optical cell sub assembly, according to one embodiment;

FIG. 6 depicts a perspective exploded view of the field-replaceable filter media of FIGS. 5A-5B, according to one embodiment;

FIG. 7 depicts a gas detection system including an optical cell assembly and installed field-replaceable filter media, according to one embodiment;

FIG. 8 depicts the field-replaceable filter media of FIGS. 5A-5B being inserted into the optical cell sub assembly during use in field, according to one embodiment;

FIG. 9A depicts the step for applying an adhesive to a backing plate to assemble the field-replaceable filter media, according to one embodiment;

FIG. 9B depicts the step for installing a filter media to the bracket to assemble the field-replaceable filter media, according to one embodiment;

FIG. 9C depicts the step for connecting the packing and filter media into the frame to assemble the field-replaceable filter media, according to one embodiment;

FIG. 9D depicts the step for connecting a dust filter gasket to the dust filter subassembly to assemble the field-replaceable filter media, according to one embodiment;

FIG. 10 depicts a high-level flowchart of a method of assembling a field-replaceable media filter and performing gas detection using a gas detection system with the field-replaceable media filter, according to one embodiment;

FIG. 11 illustrates an example top-level functional block diagram of a system including an optical cell assembly with a field-replaceable filter media, according to one embodiment;

FIG. 12 illustrates an example top-level functional block diagram of a computing device embodiment, according to one embodiment;

FIG. 13 shows a high-level block diagram and process of a computing system for implementing an embodiment of the system and process, according to one embodiment;

FIG. 14 shows a block diagram and process of an exemplary system in which an embodiment may be implemented, according to one embodiment;

FIG. 15 depicts a cloud-computing environment for implementing an embodiment of the system and process disclosed herein, according to one embodiment; and

FIG. 16 depicts a system for detecting trace gases, according to one embodiment.

### DETAILED DESCRIPTION

The present system and method allow for a set of dust filters that are configured to be replaceable or serviceable for preventing dust ingress on sensitive optics in a Tunable Diode Laser absorption spectrometer (TDLAS) or similar in-situ measurement device. The disclosed system and method include field-replaceable particle filters, or field-replaceable dust filters, for trace-gas measurement devices. The term “field-replaceable” may describe the act of replacing the filter media outside a lab or controlled manufacturing environment. The benefit of using a field-replaceable particle filter is that as the filters become inundated with particulate matter and sensor reaction time increases (increase in measurement hysteresis or complete restriction of flow), the sensor operators can replace the filter media for a ‘clean’ and unsoiled filter media.

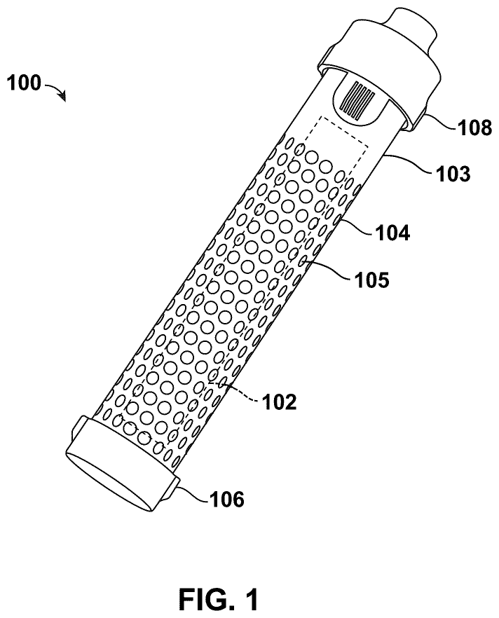

FIG. 1 depicts an optical cell assembly 100 of a gas detection system, according to one embodiment. The optical cell assembly 100 may include an optical core based trace gas sensor 102 and an optical cell sub assembly 103, or protective housing, configured to house the optical core based trace gas sensor 102 to prevent any damage to the gas sensor 102 and electronics. The optical cell sub assembly 103 of optical cell assembly 100 may include a field-replaceable filter media, such as a perforated outer sheath 104, and end caps 106, 108. The perforated outer sheath 104 may have a shape surrounding at least portion of the optical core based trace gas sensor 102 and include a plurality of apertures 105 that protects the optical core based trace gas sensor 102 from damaging impacts, deflects debris, and allows for ample airflow to pass through the gas sensor 102. In some embodiments, the perforated outer sheath 104 may be a perforated cover or wrapping element, which is configured to surround at least portion of the optical core. In some embodiments, the perforated outer sheath 104 may include a cloth mesh and/or a fine metal mesh, which may be used to reduce water ingress to allow gas flow. In some embodiments, the perforated outer sheath 104 may have a cylindrical shape with a plurality of apertures 105 arranged along at least a portion of its circumference. The end caps 106, 108 contain the optical core based trace gas sensor 102 and electronics within the optical cell sub assembly 103. In some embodiments, the perforated outer sheath 104 may be detachably attached to the end caps 106, 108 such that the perforated outer sheath 104, end caps 106, 108, and/or optical core may be replaced independently.

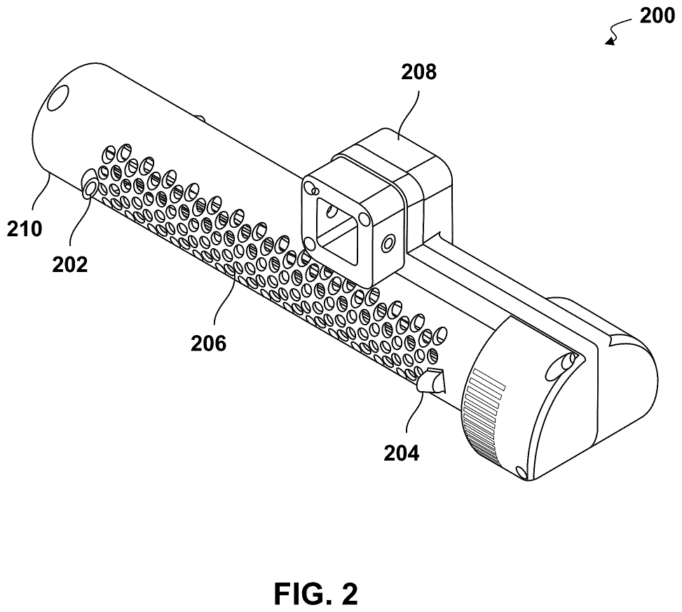

FIG. 2 depicts an optical cell assembly 200 of a gas detection system, according to one embodiment. The optical cell assembly 200 may include an optical core based trace gas sensor contained inside and an optical cell sub assembly 210 configured to house the optical core based trace gas sensor. In some embodiments, the optical cell sub assembly 210 may be an optical head enclosure with one or more imbedded nozzles 202, 204. There exists a need to clean particulate matter that collects on sensitive optical core, or optics, within the trace gas sensor gently and noninvasively. The trace gas sensor may include one or more mirrors, such as in an open path Herriot cell optics. The reflective surface of the mirrors may be easily scratched by foreign bodies. Cleaning the mirrors in an external environment, such as an oil field, is challenging. The surface of the optical head enclosure 210 may be a field-replaceable filter media including one or more apertures 206 or openings for allowing ambient gas to enter the sensor disposed in the interior of the optical head enclosure 200. During use, such as in an oil field, the optics of the sensor may become dirty, covered with dust, or the like. Dust may impede the accuracy of the sensor to detect trace-gasses. Regular cleaning of the optics of the sensor may ensure that the trace-gas detection is accurate and allow for prolonged use of the sensor. The optical head enclosure 200 may be attached to a handle, aerial vehicle, unmanned aerial vehicle (UAV), or the like, via one or more enclosure attachments 208. The one or more imbedded nozzles 202, 204 may be disposed on and through the surface of the optical head enclosure 210 to allow for cleaning of the optics of the sensor within the optical head enclosure 210. In some embodiments, the surface of the optical head enclosure 210 may be detachably attached to the one or more enclosure attachments 208 and/or one or more imbedded nozzles 202, 204 such that the surface of the optical head enclosure 210 may be replaced independently.

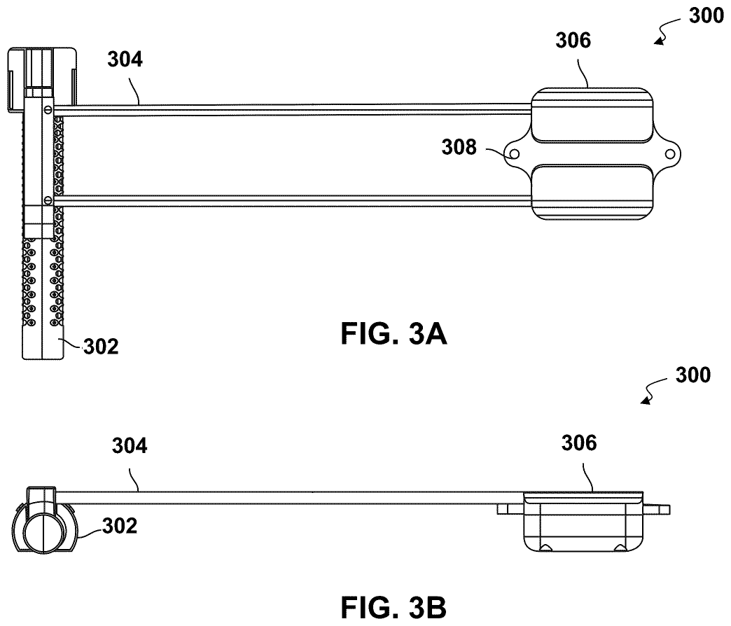

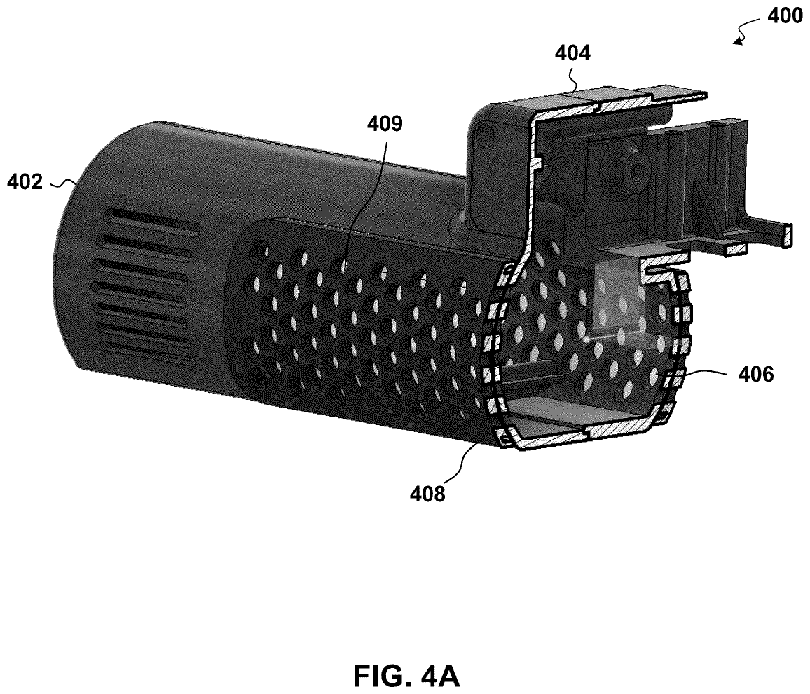

With respect to FIGS. 3A and 3B, a top perspective view and a side perspective view of a gas detection system 300 are shown, respectively. The gas detection system 300 may comprise an optical cell assembly 302, one or more extenders 304, control electronics 306, and/or one or more mounting attachments 308. The optical cell assembly 302 may include an optical core based trace gas sensor contained inside and an optical cell sub assembly having a shape surrounding the optical core based trace gas sensor. In some embodiments, the optical cell sub assembly may include a housing with at least one opening or aperture and a field-replaceable filter media disposed on the opening or aperture of the housing. The housing, or cover, of the optical cell sub assembly may have a shape surrounding at least portion of the optical core based trace gas sensor, and the field-replaceable filter media may configured to be detachably attached to a portion of the housing. The field-replaceable filter media may be further configured to protect the optical core based trace gas sensor from dust entering into the optical cell sub assembly, while allowing gas flow into the optical cell sub assembly. The optical core based trace gas sensor may be configured to measure ambient trace gas concentrations of at least one of the following gasses: methane, ethane, propane, butane, and/or natural gas. The optical cell sub assembly of the optical cell assembly 302 may include air holes for allowing air into the optical core based trace gas sensor for detecting trace gas concentrations while protecting the trace gas sensor from impacts, dust, and the like. The extenders 304 may include one or more rods or other structures to place the optical cell assembly 302 distal from the mounting attachments 308. The extenders 304 may be used to allow the sensor of the optical cell assembly 302 to be located away from an unmanned vehicle for more accurate ambient readings of trace gas concentrations. The control electronics 306 may include a processor, addressable memory, a transmitter, a receiver, a transceiver, and/or a power source. The control electronics 306 may be electronically connected to the sensor. The mounting attachments 308 may allow the gas detection system 300 to be attached to an unmanned vehicle, such as via screws, nuts, and bolts, or the like. While one configuration is shown, other gas detection system configurations for mounting to an unmanned vehicle and/or other fixed and moveable locations are possible and contemplated.

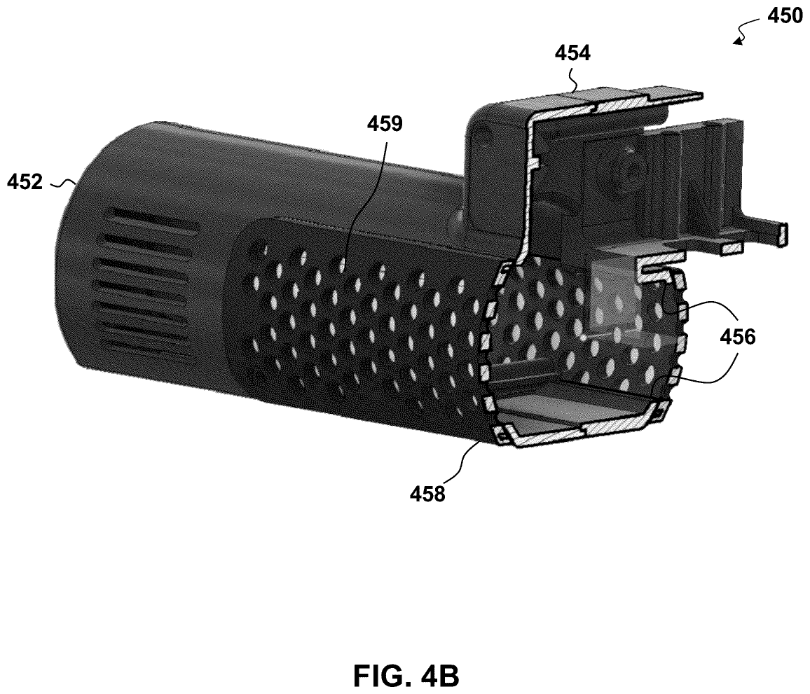

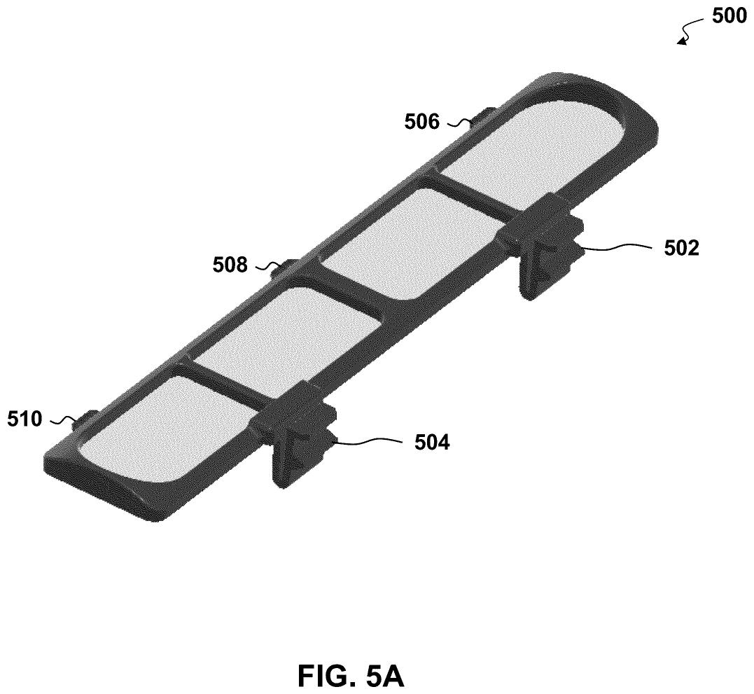

FIG. 4A depicts a partial cut-away perspective view of an optical cell sub assembly 400 having a field-replaceable filter media 408, according to one embodiment. In some embodiments, the optical cell sub assembly 400 having the field-replaceable filter media 408 may be a configuration included in the optical cell assembly 302 of the gas detection system 300 shown in FIGS. 3A and 3B but is not limited thereto. The optical cell sub assembly 400 may include a housing 402 having one or more enclosure attachments 404. The housing 402 of the optical cell sub assembly 400 may have a plurality of apertures 406 that protects the optical core based trace gas sensor from damaging impacts, deflects debris, and allows for ample airflow to pass through the sensor. The optical cell sub assembly 400 may have a field-replaceable filter media 408 disposed on the plurality of apertures 406 of the housing 402. In some embodiments, the field-replaceable filter media 408 may include a plurality of apertures 409. In this case, the field-replaceable filter media 408 may be disposed on the housing 402 such that the plurality of apertures 409 of the field-replaceable filter media 408 are respectively aligned with the plurality of apertures 406 of housing 402 but is not limited thereto. In some embodiments, a plurality of apertures 409 of the field-replaceable filter media 458 and the plurality of apertures 406 of housing 402 may be the exact same set of holes in size, shape, and pattern. In some embodiments, the housing 402 may have one big opening instead of the plurality of apertures 406, and the field-replaceable filter media 408 with the plurality of apertures 409 may be inserted into the opening of the housing 402.

FIG. 4B depicts a partial cut-away perspective view of an optical cell sub assembly 450 having a field-replaceable filter media 458, according to another embodiment. In some embodiments, the optical cell sub assembly 450 having the field-replaceable filter media 458 may be a configuration included in the optical cell assembly 302 of the gas detection system 300 shown in FIGS. 3A and 3B but is not limited thereto. Referring to FIG. 4B, the optical cell sub assembly 450 may include a housing 452 having one or more enclosure attachments 454. The housing 452 of the optical cell sub assembly 450 may have a large opening 456 that is covered by the field-replaceable filter media 458. The large opening 456 with the field-replaceable filter media 458 may allow for improved airflow to pass through the sensor while filtering dust effectively. The field-replaceable filter media 458 may be disposed on and inserted into the opening 456 of the housing 452. In some embodiments, the field-replaceable filter media 458 may include a plurality of apertures 459 but is not limited thereto.

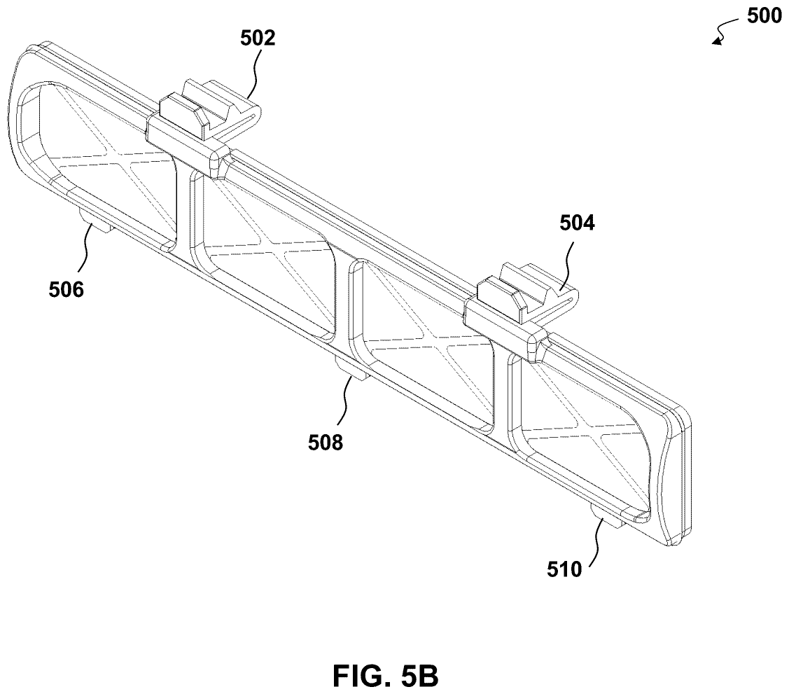

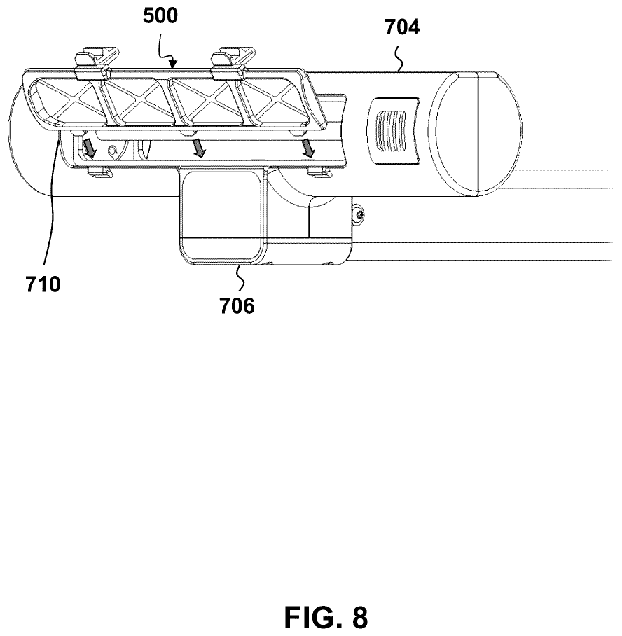

FIGS. 5A-5B depict perspective views of a field-replaceable filter media 500 for an optical cell sub assembly, according to one embodiment. In some embodiments, the field-replaceable filter media 500 may be a configuration disposed on the housing 402 of the optical cell sub assembly 400 instead of the field-replaceable filter media 408 shown in FIG. 4A but is not limited thereto. In some embodiments, the field-replaceable filter media 500 may be a configuration disposed on the housing 452 of the optical cell sub assembly 450 instead of the field-replaceable filter media 458 shown in FIG. 4B. The field-replaceable filter media 500 may include one or more clips 502, 504 to secure the field-replaceable filter media 500 to a corresponding portion or opening in a housing of an optical cell sub assembly. The field-replaceable filter media 500 may also include one or more protrusions 506, 508, 510 to align the field-replaceable filter media 500 with one or more corresponding portion or openings in a housing of the optical cell sub assembly, such as shown in FIG. 8.

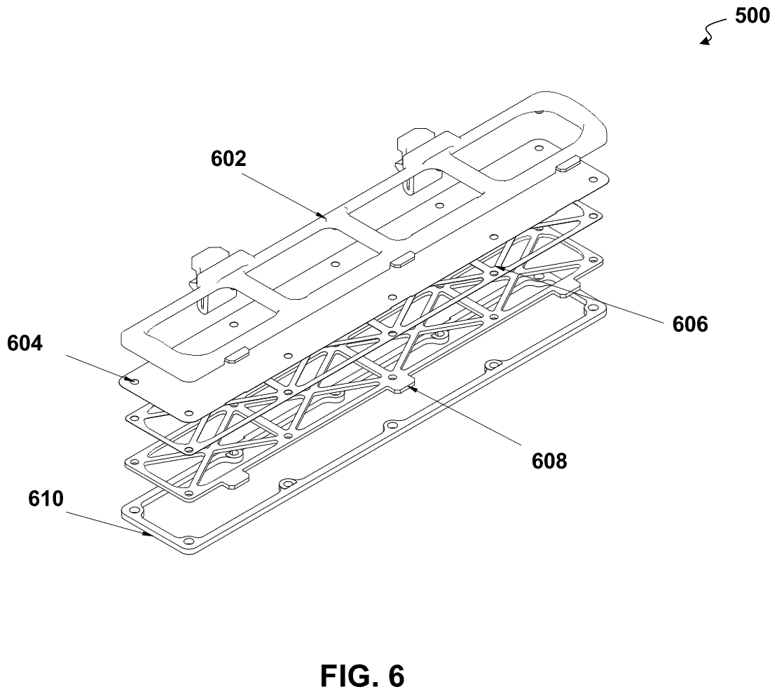

FIG. 6 depicts a perspective exploded view of the field-replaceable filter media 500 of FIGS. 5A-5B, according to one embodiment. The field-replaceable filter media 500 may include a dust filter frame 602, a filter media 604, a filter backing plate 608, a dust filter gasket 610, and a dust filter adhesive 606.

In some embodiments, the filter media 604 may be made of the material having a plurality of pores. The filter media 604 may comprise a Nylon monofilament (NMO) with a pore size of approximately 25 microns to allow ample airflow while keeping out dust particles greater than approximately 25 micron. This pore size may be selected in one embodiment based on a careful consideration of particle size ranges of common substances and dusts. In some embodiments, the filter media 604 may be made of at least one of: a polypropylene filter paper, a filter felt; a rayon/polyester fabric, a chemical resistant filter felt, a polyethersulfone (PES) membrane, and hydrophobic or oleophobic materials. In some embodiments, the dust filter adhesive 606 may be a double-sided transfer tape. In some embodiments, the filter backing plate 608 may comprise carbon fiber. In some embodiments, the dust filter frame 602 may comprise plastic. In some embodiments, the dust filter gasket 610 may be a foam sealing gasket.

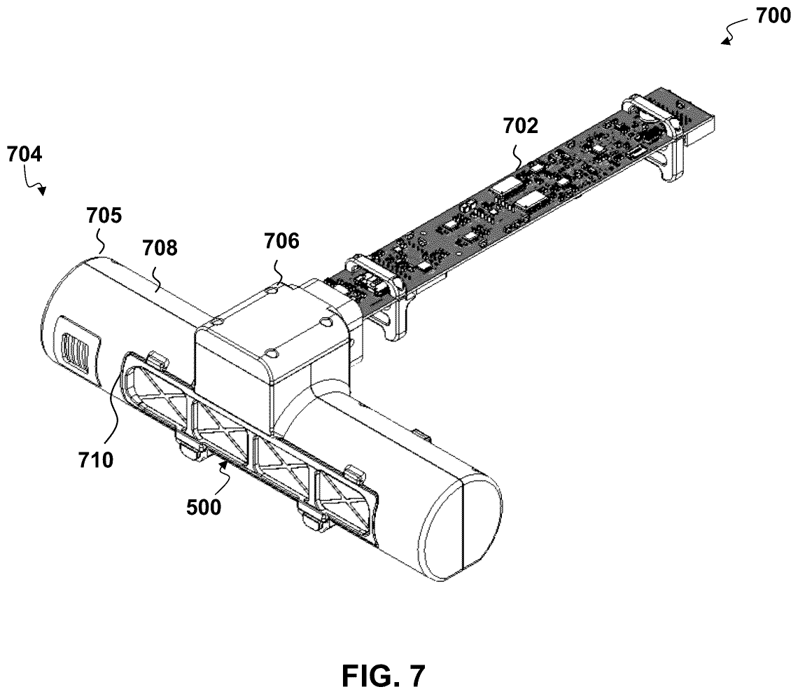

FIG. 7 depicts a gas detection system 700 including an optical cell assembly 704 and a field-replaceable filter media 500 installed on an optical cell sub assembly of the optical cell assembly 704, according to one embodiment. The gas detection system 700 may include control electronics 702. The control electronics 702 may include a processor, addressable memory, a transmitter, a receiver, a transceiver, and/or a power source. The control electronics 702 may be in contact with and/or electronically connected to an optical core based trace gas sensor inside the optical cell sub assembly of the optical cell assembly 704. Mounting attachments 706 may allow the optical cell assembly 704 to be attached to an unmanned vehicle, such as via screws, nuts, and bolts, or the like. While one configuration is shown, other configurations for mounting to an unmanned vehicle and/or other fixed and moveable locations are possible and contemplated. The field-replaceable filter media 500 may be detachably attached to a portion and/or opening 710 of a housing 708 of the optical cell assembly 704 to prevent large particles from entering into the optical cell assembly 704 while maintaining desired air flow through the optical cell assembly 704.

FIG. 8 depicts the field-replaceable filter media 500 of FIGS. 5A-5B being inserted into an opening 710 in a housing of the optical cell sub assembly 704 during use in the field, according to one embodiment. While the field-replaceable filter media 500 may include one or more protrusions (506, 508, 510, FIG. 5) and one or more clips (502, 504, FIG. 5) to attach and/or detach the field-replaceable filter media 500 from the optical cell sub assembly 704, other attachment methods are possible and contemplated.

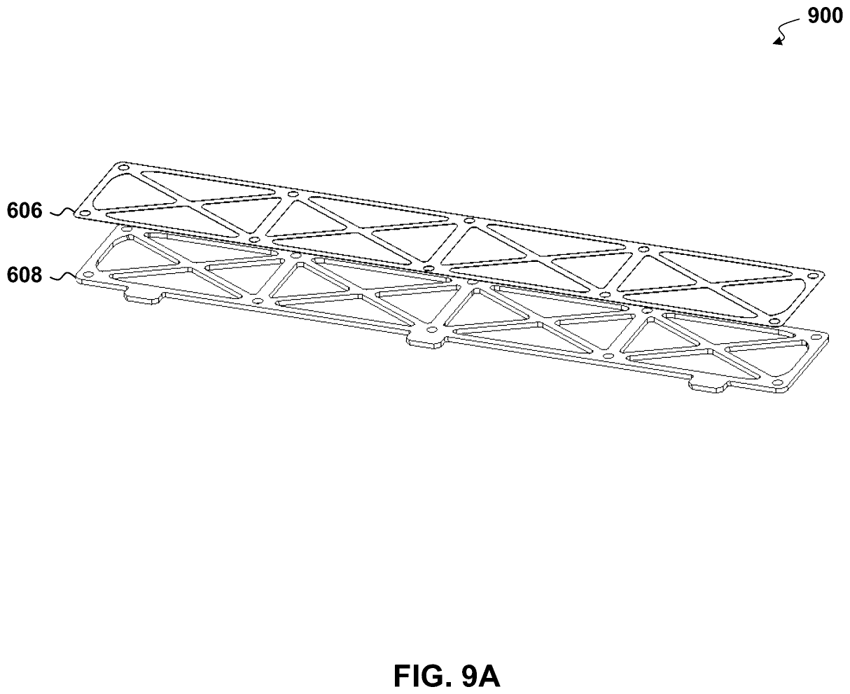

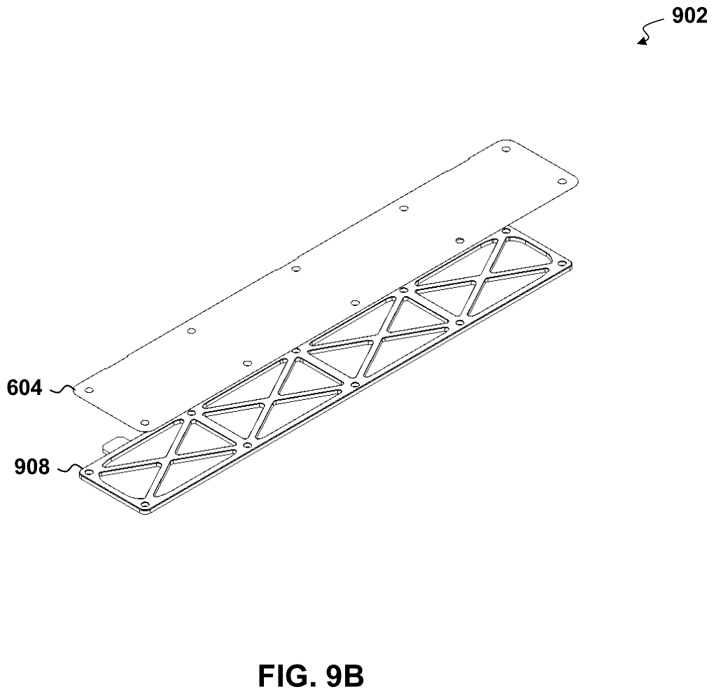

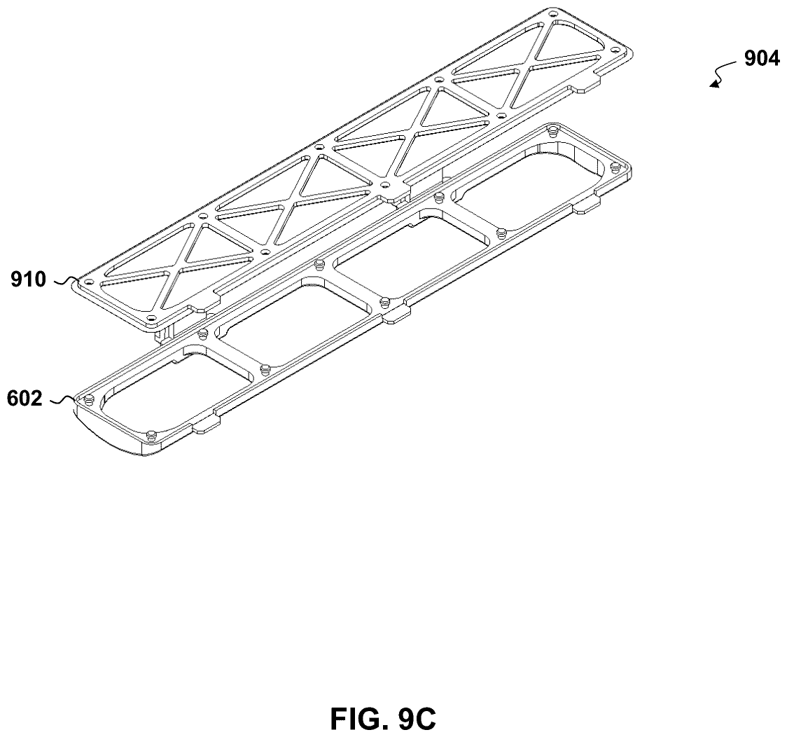

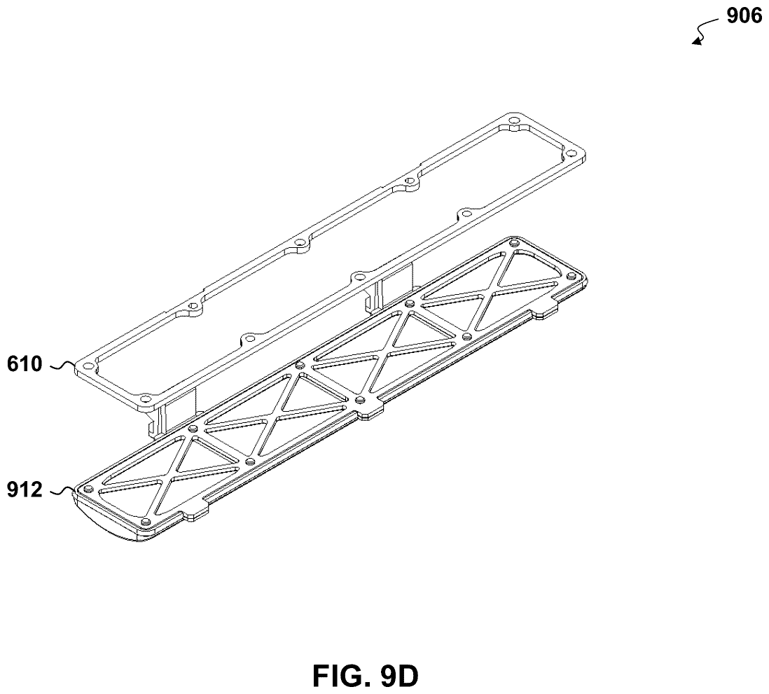

FIGS. 9A to 9D depict the method of assembling the field-replaceable filter media (500, FIG. 6), according to one embodiment. FIG. 9A depicts the step 900 for applying the dust filter adhesive 606 to the filter backing plate 608 to assemble the field-replaceable filter media (500, FIG. 6), according to one embodiment. Attaching the dust filter adhesive 606 to the filter backing plate 608 may form a bracket (908, FIG. 9B) once assembled. In some embodiments, the dust filter adhesive 606 may be a double-sided transfer tape. In some embodiments, the filter backing plate 608 may comprise a carbon fiber backing plate.

FIG. 9B depicts the step 902 for installing the filter media 604 to the bracket 908 to assemble the field-replaceable filter media (500, FIG. 6), according to one embodiment. Attaching the filter media 604 to the bracket 908 may form a packing and filter media (910, FIG. 9C) once assembled. In some embodiments, an adhesive backing may be removed from the bracket 908 and the filter media 604 may be installed using a plastic spatula or other device to remove any air bubbles and/or ripples.

FIG. 9C depicts the step 904 for connecting the packing and filter media 910 into the frame 602 to assemble the field-replaceable filter media (500, FIG. 6), according to one embodiment. Attaching the packing and filter media 910 into the frame 602 may form a dust filter subassembly (912, FIG. 9D) once assembled. In some embodiments, the packing and filter media 910 may be press fit into the frame 602.

FIG. 9D depicts the step 906 for connecting the dust filter gasket 610 to the dust filter subassembly 912 to assemble the field-replaceable filter media (500, FIG. 6), according to one embodiment. Attaching the dust filter gasket 610 to the dust filter subassembly 912 may form a dust filter assembly once assembled. The dust filter assembly may be used by a user as a field-replaceable filter media for an optical cell assembly. In some embodiments, an adhesive backing from the foam sealing gasket may be removed and pressed into the dust filter subassembly 912.

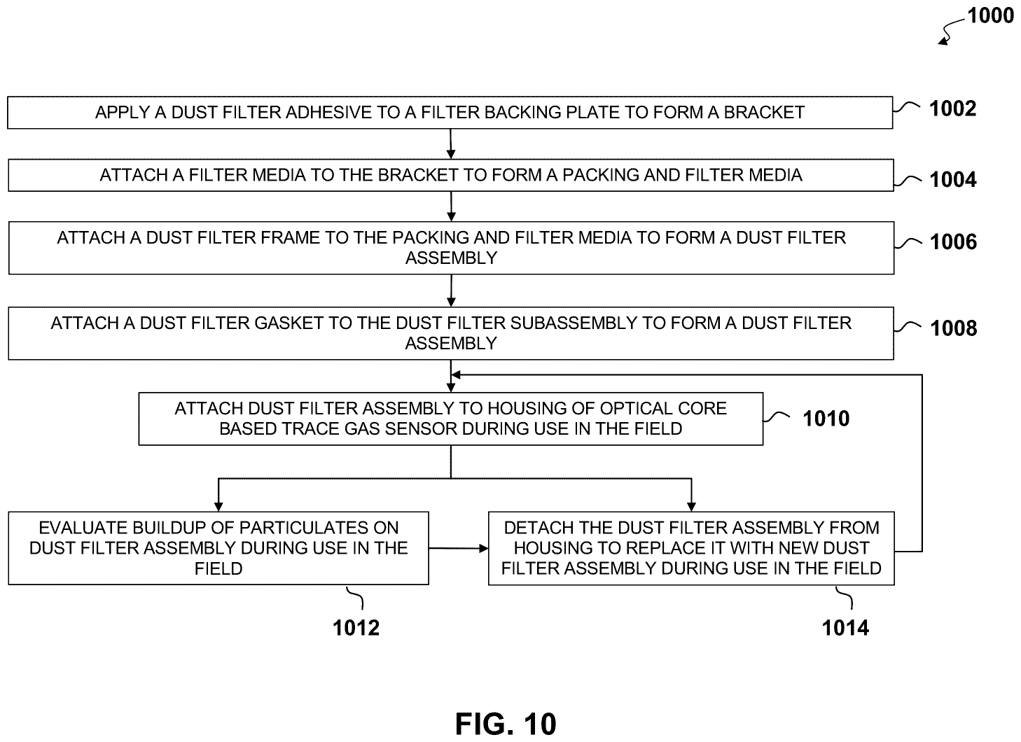

FIG. 10 depicts a high-level flowchart of a method 1000 of assembling a field-replaceable media filter and performing gas detection using a gas detection system with the field-replaceable media filter, according to one embodiment. The method 1000 may include applying a dust filter adhesive to a filter backing plate to form a bracket (step 1002). The method 1000 may then include attaching a filter media to the bracket to form a packing and filter media (step 1004). The method 1000 may then include attaching a dust filter frame to the packing and filter media to form a dust filter subassembly (step 1006). The method may then include attaching a dust filter gasket to the dust filter subassembly to form a dust filter assembly (step 1008). Once the dust filter assembly is prepared, the dust filter assembly may be detachably attached to a portion of the housing of an optical core based trace gas sensor in a gas detection system for a method of performing gas detection using a gas detection system with a dust filter assembly. That is, the method of performing gas detection may comprise: attaching the dust filter assembly into an opening of the housing of the optical core based trace gas sensor in the gas detection system during use in the field (step 1010); and detaching the dust filter assembly from the housing to replace it with a new dust filter assembly during use in the field (step 1014). In some embodiments, the method of performing gas detection may further comprise evaluating a buildup of particulates on the dust filter assembly during use in the field (step 1012). The step of evaluating a buildup of particulates on the dust filter assembly may be performed based on at least one of: a visual inspection of the dust filter assembly using a digital image processing module of the gas detection system; and a pressure drop measurement across the dust filter assembly using a pressure drop measurement module of the gas detection system.

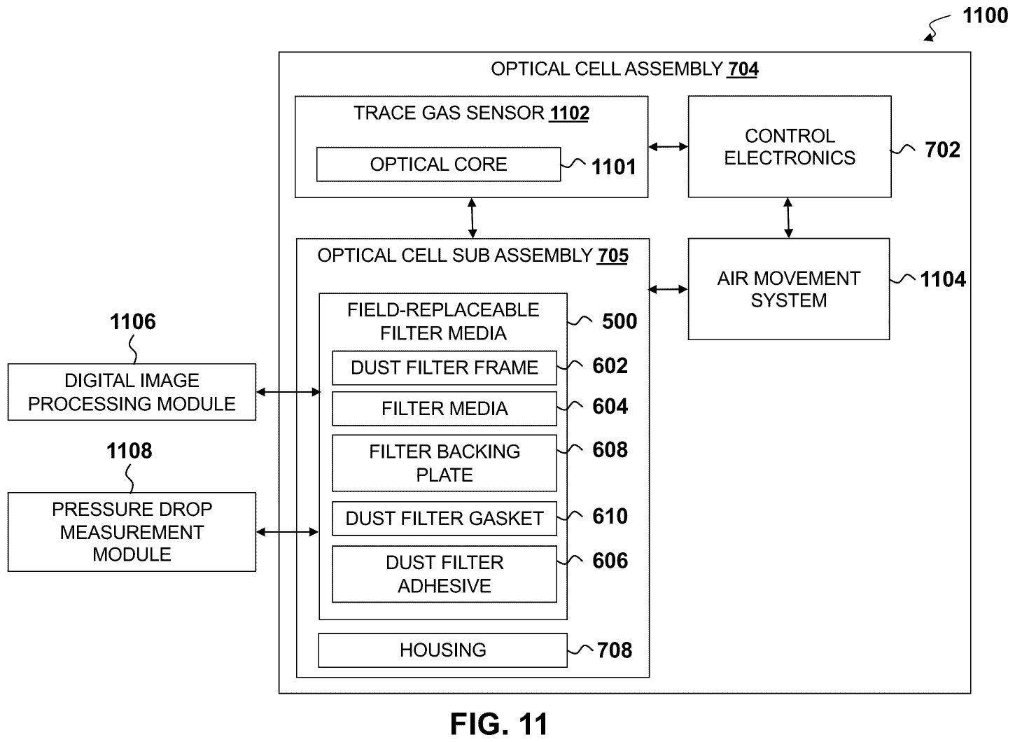

FIG. 11 illustrates an example top-level functional block diagram of a gas detection system 1100 including an optical cell assembly 704 with a field-replaceable filter media 500, according to one embodiment. The optical cell assembly 704 may include a trace gas sensor 1102 with an optical core 1101, an optical cell sub assembly 705, control electronics 702, and/or an air movement system 1104. The optical cell sub assembly 705 may include a housing 708 and a field-replaceable filter media 500 and have a shape to surround the trace gas sensor 1102 with the optical core 1101.

As mentioned above with reference to FIGS. 6, 9A to 10, the field-replaceable filter media 500 may include the dust filter frame 602, the filter media 604, the filter backing plate 608, the dust filter gasket 610, and the dust filter adhesive 606. Multiple materials for the filter media 604 may be used to achieve a desired result. In some embodiments, the filter media 604 may be made of the material having a plurality of pores. In this case, if the pore sizes of the filter media 604 are below a predetermined level (e.g., less than approximately 25 microns in the filter media 604), then the optical cell assembly 704 may need to be paired with some type of forced air solution, such as an air movement system 1104, pulling the gas into the sample chamber inside the optical cell sub assembly 705, and expelling it at a constant rate. This air movement system 1104 may include: a pump system, a small fan or series of fans, a solid state device, or the like.

The field-replaceable filter media 500 may be detachably attached to a portion of the housing 708 of the optical cell assembly 704 to prevent large particles from entering into the optical cell assembly 704 while maintaining desired air flow through the optical cell assembly 704.

The filter media 604 may filter particle sizes greater than approximately 25 micrometers in diameter. Other particle sizes may be filtered based on the desired use. In some embodiments, particles of any size may be filtered, not to exceed approximately 2.54 cm in diameter. Particle sizes may vary from approximately 44 micrometers down to 0.2 micrometers, in some embodiments. A pore size of approximately 25 micrometers may provide a low flow restriction while reducing the amount of the amount of dust that is present in industrial energy, agricultural, or landfill environments. Particles may be solids (e.g., dust, rocks, shavings, skin cells, etc.) or liquids (e.g., water in the form of droplets, stream, fog, rain, water vapor, etc.).

The filter media 604 may comprise a Nylon monofilament (NMO) with a pore size of approximately 25 microns to allow ample airflow while keeping out dust particles greater than approximately 25 micron. This pore size may be selected in one embodiment based on a careful consideration of particle size ranges of common substances and dusts. Smaller pore sizes, such as approximately 1 micron, may result in an increased resident time of gas in the system, effectively trapping the gas inside the sample chamber inside the optical cell sub assembly 705 for long periods of time.

In some embodiments, a nylon monofilament (NMO) may have a pore size between approximately 5 and 25 microns; a polypropylene filter paper may have a pore size of approximately 40 microns; a filter felt may have a pore size of approximately 25 microns; a rayon/polyester fabric may have a pore size of approximately 44 microns; a chemical resistant filter felts may have a pore size of approximately 25 microns; a polyethersulfone (PES) membrane may have a pore size of approximately 0.2 microns, 1.2 microns, 3 microns, and/or 5 microns.

In some embodiments, the filter media 604 may target hydrophobic or oleophobic materials in order to increase an Ingress Protection (IP) rating of the gas detection system 1100 and try and keep out excess moisture when used in harsh conditions.

In some embodiments, the gas detection system 1100 may further include a digital image processing module 1106 and a pressure drop measurement 1108. The digital image processing 1106 may be used to determine when the field-replaceable filter media 500 needs to be replaced.

The pressure drop measurement module 1108 may be used to determine when the field-replaceable filter media 500 needs to be replaced.

The field-replaceable filter media 500 may be replaced on a service interval of every three months or sooner based on a visual inspection of the field-replaceable filter media 500. This evaluation of the field-replaceable filter media 500 could be done using the digital image processing module 1106 or some other advanced method to evaluate the field-replaceable filter media 500. The processed image obtained from the digital image processing module 1106 may be used to determine when the field-replaceable filter media 500 needs to be replaced. The pressure drop measurement module 1108 may measure a pressure drop across the field-replaceable filter media 500, and the pressure drop measurement obtained from the pressure drop measurement module 1108 may be used to evaluate a buildup of particulates on the filter media 604 and/or the field-replaceable filter media 500.

In some embodiments, one or more components of the field-replaceable filter media 500 may be reusable. The field-replaceable filter media 500 may go through a sterilization process in order to reduce an environmental impact.

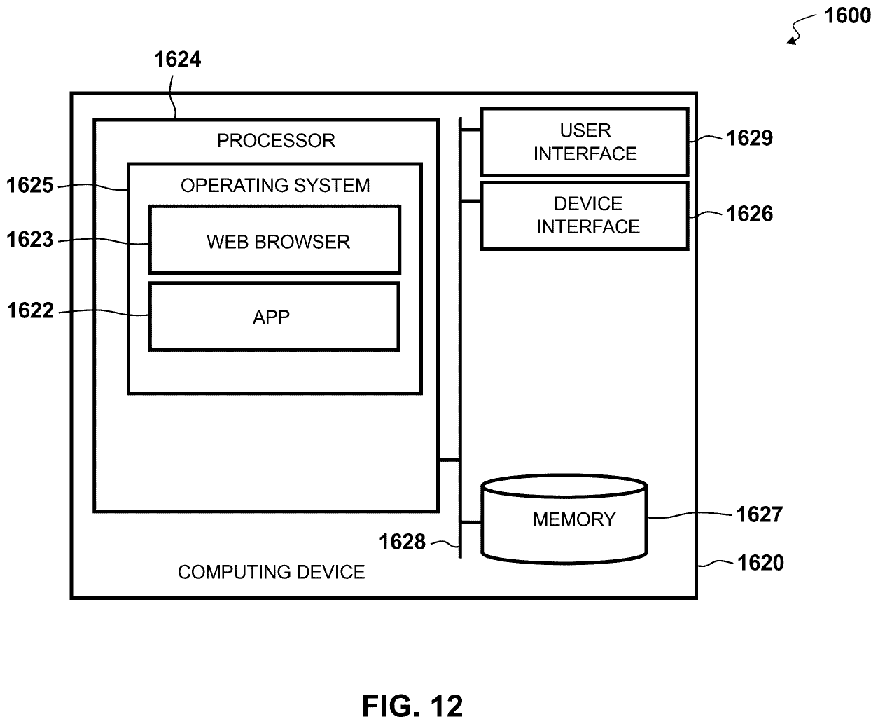

FIG. 12 illustrates an example of a top-level functional block diagram of a computing device embodiment 1600. The example operating environment is shown as a computing device 1620 comprising a processor 1624, such as a central processing unit (CPU), addressable memory 1627, an external device interface 1626, e.g., an optional universal serial bus port and related processing, and/or an Ethernet port and related processing, and an optional user interface 1629, e.g., an array of status lights and one or more toggle switches, and/or a display, and/or a keyboard and/or a pointer-mouse system and/or a touch screen. Optionally, the addressable memory may, for example, be: flash memory, EPROM, and/or a disk drive or other hard drive. These elements may be in communication with one another via a data bus 1628. In some embodiments, via an operating system 1625 such as one supporting a web browser 1623 and applications 1622, the processor 1624 may be configured to execute steps of a process establishing a communication channel and processing according to the embodiments described above.

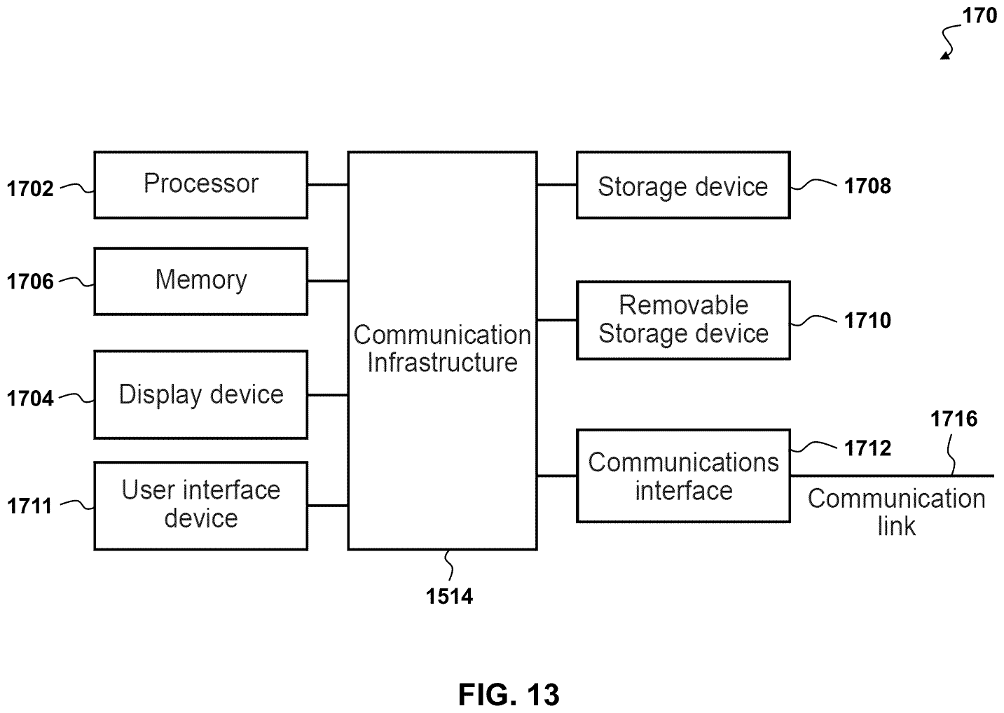

FIG. 13 is a high-level block diagram 1700 showing a computing system comprising a computer system useful for implementing an embodiment of the system and process, disclosed herein. Embodiments of the system may be implemented in different computing environments. The computer system includes one or more processors 1702, and can further include an electronic display device 1704 (e.g., for displaying graphics, text, and other data), a main memory 1706 (e.g., random access memory (RAM)), storage device 1708, a removable storage device 1710 (e.g., removable storage drive, a removable memory module, a magnetic tape drive, an optical disk drive, a computer readable medium having stored therein computer software and/or data), user interface device 1711 (e.g., keyboard, touch screen, keypad, pointing device), and a communication interface 1712 (e.g., modem, a network interface (such as an Ethernet card), a communications port, or a PCMCIA slot and card). The communication interface 1712 allows software and data to be transferred between the computer system and external devices. The system further includes a communications infrastructure 1514 (e.g., a communications bus, cross-over bar, or network) to which the aforementioned devices/modules are connected as shown.

Information transferred via communications interface 1712 may be in the form of signals such as electronic, electromagnetic, optical, or other signals capable of being received by communications interface 1724, via a communication link 1716 that carries signals and may be implemented using wire or cable, fiber optics, a phone line, a cellular/mobile phone link, a radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface 1712. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

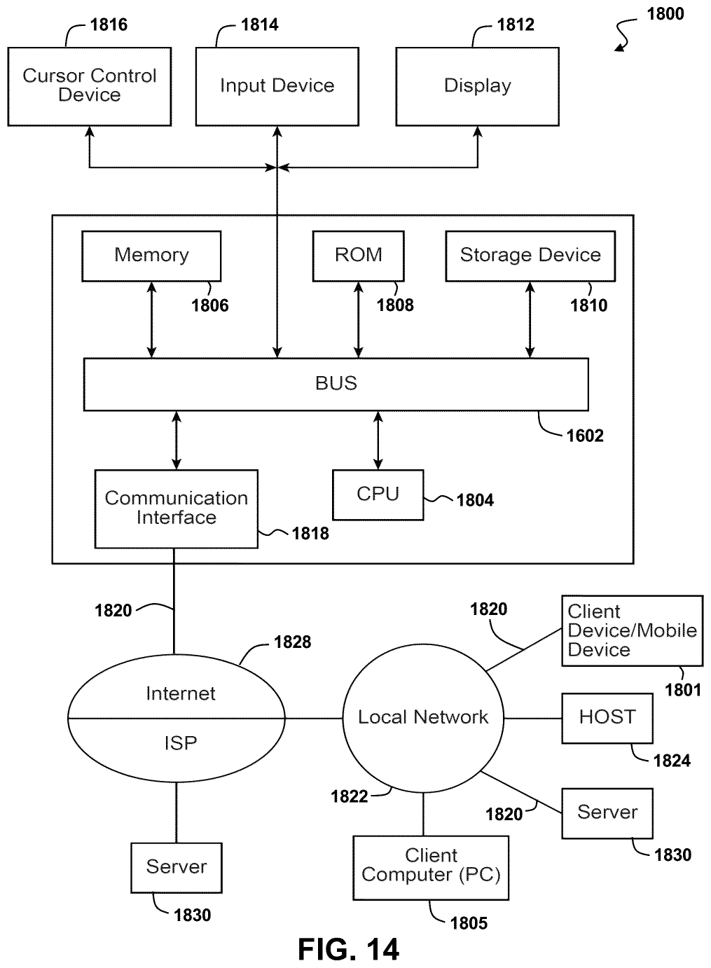

FIG. 14 shows a block diagram of an example system 1800 in which an embodiment may be implemented. The system 1800 includes one or more client devices 1801 such as consumer electronics devices, connected to one or more server computing systems 1830. A server 1830 includes a bus 1602 or other communication mechanism for communicating information, and a processor (CPU) 1804 coupled with the bus 1602 for processing information. The server 1830 also includes a main memory 1806, such as a random access memory (RAM) or other dynamic storage device, coupled to the bus 1602 for storing information and instructions to be executed by the processor 1804. The main memory 1806 also may be used for storing temporary variables or other intermediate information during execution or instructions to be executed by the processor 1804. The server computer system 1830 further includes a read only memory (ROM) 1808 or other static storage device coupled to the bus 1602 for storing static information and instructions for the processor 1804. A storage device 1810, such as a magnetic disk or optical disk, is provided and coupled to the bus 1602 for storing information and instructions. The bus 1602 may contain, for example, thirty-two address lines for addressing video memory or main memory 1806. The bus 1602 can also include, for example, a 32-bit data bus for transferring data between and among the components, such as the CPU 1804, the main memory 1806, video memory and the storage 1810. Alternatively, multiplex data/address lines may be used instead of separate data and address lines.

The server 1830 may be coupled via the bus 1602 to a display 1812 for displaying information to a computer user. An input device 1814, including alphanumeric and other keys, is coupled to the bus 1602 for communicating information and command selections to the processor 1804. Another type or user input device comprises cursor control 1816, such as a mouse, a trackball, or cursor direction keys for communicating direction information and command selections to the processor 1804 and for controlling cursor movement on the display 1812.

According to one embodiment, the functions are performed by the processor 1804 executing one or more sequences of one or more instructions contained in the main memory 1806. Such instructions may be read into the main memory 1806 from another computer-readable medium, such as the storage device 1810. Execution of the sequences of instructions contained in the main memory 1806 causes the processor 1804 to perform the process steps described herein. One or more processors in a multi-processing arrangement may also be employed to execute the sequences of instructions contained in the main memory 1806. In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 1804 for execution. Such a medium may take many forms, including but not limited to, non-volatile media, volatile media, and transmission media. Non-volatile media includes, for example, optical or magnetic disks, such as the storage device 1810. Volatile media includes dynamic memory, such as the main memory 1806. Transmission media includes coaxial cables, copper wire and fiber optics, including the wires that comprise the bus 1602. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 1804 for execution. For example, the instructions may initially be carried on a magnetic disk of a remote computer. The remote computer can load the instructions into its dynamic memory and send the instructions over a telephone line using a modem. A modem local to the server 1830 can receive the data on the telephone line and use an infrared transmitter to convert the data to an infrared signal. An infrared detector coupled to the bus 1602 can receive the data carried in the infrared signal and place the data on the bus 1602. The bus 1602 carries the data to the main memory 1806, from which the processor 1804 retrieves and executes the instructions. The instructions received from the main memory 1806 may optionally be stored on the storage device 1810 either before or after execution by the processor 1804.

The server 1830 also includes a communication interface 1818 coupled to the bus 1602. The communication interface 1818 provides a two-way data communication coupling to a network link 1820 that is connected to the world wide packet data communication network now commonly referred to as the Internet 1828. The Internet 1828 uses electrical, electromagnetic, or optical signals that carry digital data streams. The signals through the various networks and the signals on the network link 1820 and through the communication interface 1818, which carry the digital data to and from the server 1830, are exemplary forms or carrier waves transporting the information.

In another embodiment of the server 1830, the communication interface 1818 is connected to a network 1822 via a communication link 1820. For example, the communication interface 1818 may be an integrated services digital network (ISDN) card or a modem to provide a data communication connection to a corresponding type of telephone line, which can comprise part of the network link 1820. As another example, the communication interface 1818 may be a local area network (LAN) card to provide a data communication connection to a compatible LAN. Wireless links may also be implemented. In any such implementation, the communication interface 1818 sends and receives electrical, electromagnetic, or optical signals that carry digital data streams representing various types of information.

The network link 1820 typically provides data communication through one or more networks to other data devices. For example, the network link 1820 may provide a connection through the local network 1822 to a host computer 1824 or to data equipment operated by an Internet Service Provider (ISP). The ISP in turn provides data communication services through the Internet 1828. The local network 1822 and the Internet 1828 both use electrical, electromagnetic, or optical signals that carry digital data streams. The signals through the various networks and the signals on the network link 1820 and through the communication interface 1818, which carry the digital data to and from the server 1830, are exemplary forms or carrier waves transporting the information.

The server 1830 can send/receive messages and data, including e-mail, program code, through the network, the network link 1820 and the communication interface 1818. Further, the communication interface 1818 can comprise a USB/Tuner and the network link 1820 may be an antenna or cable for connecting the server 1830 to a cable provider, satellite provider or other terrestrial transmission system for receiving messages, data and program code from another source.

The example versions of the embodiments described herein may be implemented as logical operations in a distributed processing system such as the system 1800 including the servers 1830. The logical operations of the embodiments may be implemented as a sequence of steps executing in the server 1830, and as interconnected machine modules within the system 1800. The implementation is a matter of choice and can depend on performance of the system 1800 implementing the embodiments. As such, the logical operations constituting said example versions of the embodiments are referred to for e.g., as operations, steps or modules.

Similar to a server 1830 described above, a client device 1801 can include a processor, memory, storage device, display, input device and communication interface (e.g., e-mail interface) for connecting the client device to the Internet 1828, the ISP, or LAN 1822, for communication with the servers 1830.

The system 1800 can further include computers (e.g., personal computers, computing nodes) 1805 operating in the same manner as client devices 1801, where a user can utilize one or more computers 1805 to manage data in the server 1830.

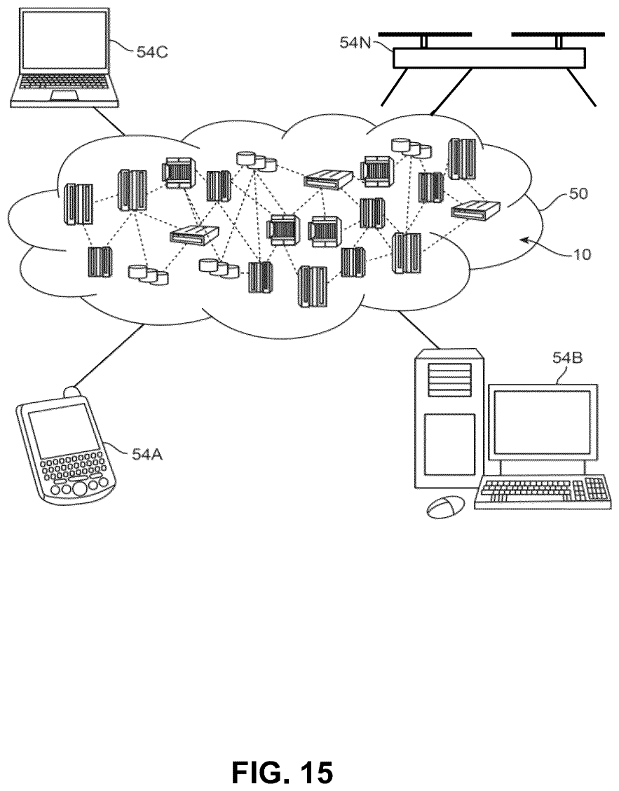

Referring now to FIG. 15, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 54B, laptop computer 54C, and/or unmanned aerial system (UAS) 54N may communicate. The nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 15 are intended to be illustrative only and that computing nodes 10 and a cloud computing environment 50 can communicate with any type of computerized device over any type of network and/or network addressable connection (e.g., using a web browser).

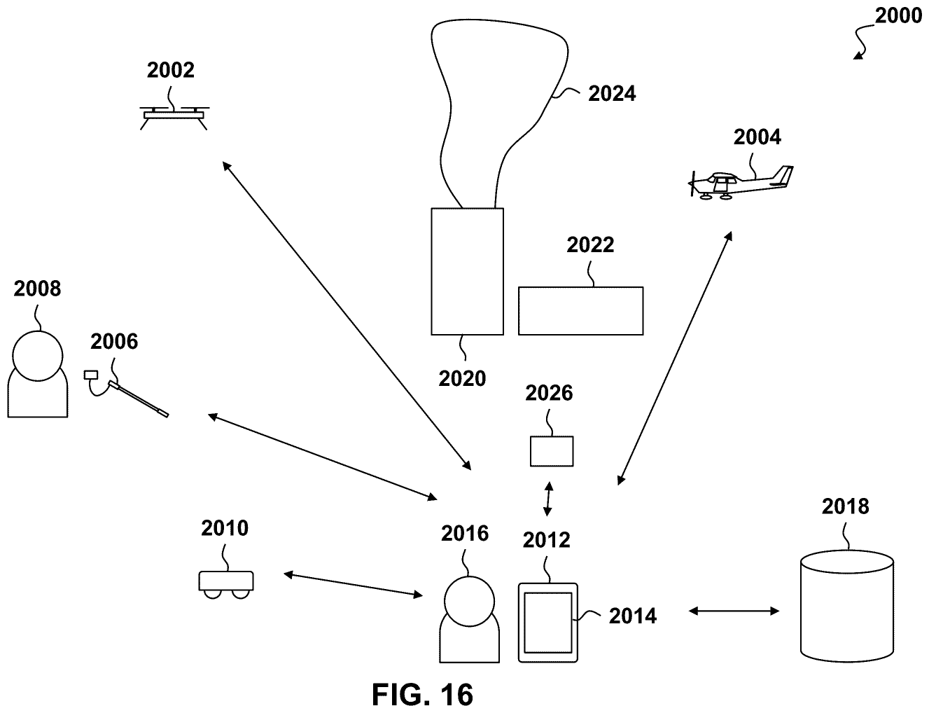

FIG. 16 depicts a system 2000 for detecting trace gases, according to one embodiment. The system may include one or more trace gas sensors located in one or more vehicles 2002, 2004, 2006, 2010. The one or more trace gas sensors may detect elevated trace gas concentrations from one or more potential gas sources 2020, 2022, such as a holding tank, pipeline, or the like. The potential gas sources 2020, 2022 may be part of a large facility, a small facility, or any location. The potential gas sources 2020, 2022 may be clustered and/or disposed distal from one another. The one or more trace gas sensors may be used to detect and quantify leaks of toxic gases, e.g., hydrogen disulfide, or environmentally damaging gases, e.g., methane, sulfur dioxide) in a variety of industrial and environmental contexts. Detection and quantification of these leaks are of interest to a variety of industrial operations, such as oil and gas, chemical production, and painting. Detection and quantification of leaks is also of value to environmental regulators for assessing compliance and for mitigating environmental and safety risks. In some embodiments, the at least one trace gas sensor may be configured to detect methane. In other embodiments, the at least one trace gas sensor may be configured to detect sulfur oxide, such as SO, SO2, SO3, S7O2, S6O2, S2O2, and the like. A trace gas leak 2024 may be present in a potential gas source 2020. The one or more trace gas sensors may be used to identify the trace gas leak 2024 and/or the source 2020 of the trace gas leak 2024 so that corrective action may be taken.

The one or more vehicles 2002, 2004, 2006, 2010 may include an unmanned aerial vehicle (UAV) 2002, an aerial vehicle 2004, a handheld device 2006, and a ground vehicle 2010. In some embodiments, the UAV 2002 may be a quadcopter or other device capable of hovering, making sharp turns, and the like. In other embodiments, the UAV 2002 may be a winged aerial vehicle capable of extended flight time between missions. The UAV 2002 may be autonomous or semi-autonomous in some embodiments. In other embodiments, the UAV 2002 may be manually controlled by a user. The aerial vehicle 2004 may be a manned vehicle in some embodiments. The handheld device 2006 may be any device having one or more trace gas sensors operated by a user 2008. In one embodiment, the handheld device 2006 may have an extension for keeping the one or more trace gas sensors at a distance from the user 2008. The ground vehicle 2010 may have wheels, tracks, and/or treads in one embodiment. In other embodiments, the ground vehicle 2010 may be a legged robot. In some embodiments, the ground vehicle 2010 may be used as a base station for one or more UAVs 2002. In some embodiments, one or more aerial devices, such as the UAV 2002, a balloon, or the like, may be tethered to the ground vehicle 2010. In some embodiments, one or more trace gas sensors may be located in one or more stationary monitoring devices 2026. The one or more stationary monitoring devices may be located proximate one or more potential gas sources 2020, 2022. In some embodiments, the one or more stationary monitoring devices may be relocated.

The one or more vehicles 2002, 2004, 2006, 2010 and/or stationary monitoring devices 2026 may transmit data including trace gas data to a ground control station (GCS) 2012. The GCS may include a display 2014 for displaying the trace gas concentrations to a GCS user 2016. The GCS user 2016 may be able to take corrective action if a gas leak 2024 is detected, such as by ordering a repair of the source 2020 of the trace gas leak. The GCS user 2016 may be able to control movement of the one or more vehicles 2002, 2004, 2006, 2010 in order to confirm a presence of a trace gas leak in some embodiments.

In some embodiments, the GCS 2012 may transmit data to a cloud server 2018. In some embodiments, the cloud server 2018 may perform additional processing on the data. In some embodiments, the cloud server 2018 may provide third party data to the GCS 2012, such as wind speed, temperature, pressure, weather data, or the like.

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention is herein disclosed by way of examples and should not be limited by the particular disclosed embodiments described above.

What is claimed is:

1. A system including an optical cell assembly, comprising:
an optical core based trace gas sensor configured to measure trace gas concentrations; and
an optical cell sub assembly including:
a housing configured to house the optical core based trace gas sensor; and
a field-replaceable filter media configured to be detachably attached to a portion of the housing and allow ambient trace gas to enter into the optical cell sub assembly.

2. The system of claim 1, wherein the field-replaceable filter media is a perforated outer sheath including a plurality of apertures.

3. The system of claim 2, wherein the perforated outer sheath has a cylindrical shape with the plurality of apertures arranged along the circumference of the cylindrical shape.

4. The system of claim 1, further comprising one or more imbedded nozzles disposed on and through the surface of the optical cell sub assembly to allow for cleaning of the optics of the optical core based trace gas sensor within the optical cell sub assembly.

5. The system of claim 1, further comprising:
one or more extenders configured to connect between the optical cell assembly and one or more mounting attachments such that the optical cell assembly is located distal from the one or more mounting attachments; and
the one or more mounting attachments configured to attach the system to an unmanned vehicle.

6. The system of claim 1, wherein the housing includes a plurality of apertures, and wherein the field-replaceable filter media is disposed on the plurality of apertures of the housing.

7. The system of claim 6, wherein the field-replaceable filter media includes a plurality of apertures, and wherein the plurality of apertures of the field-replaceable filter media are respectively aligned with the plurality of apertures of housing.

8. The system of claim 1, wherein the housing includes an opening, and wherein the field-replaceable filter media is inserted into the opening during use in the field.

9. The system of claim 8, wherein the field-replaceable filter media includes at least one of: a clip for securing the field-replaceable filter media (500) to the opening and a protrusion for aligning the field-replaceable filter media with the opening.

10. The system of claim 1, wherein the field-replaceable filter media includes:
a filter media and at least one of: a dust filter frame, a filter backing plate, and a dust filter gasket.

11. The system of claim 10, wherein the filter media is made of material having a plurality of pores.

12. The system of claim 11, when the sizes of the pores are below a predetermined level, further comprising an air movement system configured to pull ambient gas into the optical cell sub assembly and expel gas inside the optical cell sub assembly at a constant rate.

13. The system of claim 10, wherein the filter media comprise at least one of: Nylon monofilament (NMO), polypropylene filter paper, a filter felt, a rayon fabric, a polyester fabric, a chemical resistant filter felt, a polyethersulfone (PES) membrane, and hydrophobic or oleophobic materials.

14. The system of claim 1, further comprising a pressure drop measurement module configured to measure a pressure drop across the field-replaceable filter media, and wherein the pressure drop measurement obtained from the pressure drop measurement module is used to evaluate a buildup of particulates on the field-replaceable filter media.

15. The system of claim 1, further comprising a digital image processing module configured to perform a visual inspection of the field-replaceable filter media, and wherein the processed image obtained from the digital image processing module is used to determine when the field-replaceable filter media needs to be replaced.

16. The system of claim 1, further comprising control electronics electronically connected to the optical core based trace gas sensor inside the optical cell sub assembly.

17. A method comprising:
applying a dust filter adhesive to a filter backing plate to form a bracket;
attaching a filter media to the bracket to form a packing and filter media;
attaching a dust filter frame to the packing and filter media to form a dust filter subassembly; and
attaching a dust filter gasket to the dust filter subassembly to form a dust filter assembly.

18. The method of claim 17, further comprising:
attaching the dust filter assembly into an opening of a housing of an optical core based trace gas sensor in a gas detection system during use in the field; and
detaching the dust filter assembly from the housing to replace it with a new dust filter assembly during use in the field.

19. The method of claim 18, further comprising:
evaluating a buildup of particulates on the dust filter assembly based on at least one of:
a visual inspection of the dust filter assembly using a digital image processing module of the gas detection system; and
a pressure drop measurement across the dust filter assembly using a pressure drop measurement module of the gas detection system.

20. A system comprising:
an optical core based trace gas sensor configured to measure trace gas concentrations; and
a housing having a cylindrical shape configured to house the optical core based trace gas sensor, wherein the housing includes a perforated outer sheath (with a plurality of apertures arranged along at least a portion of circumference of the cylindrical shape,
wherein the perforated outer sheath is configured to be detachably attached to a portion of the housing and allow ambient trace gas to enter into the optical core based trace gas sensor.
